(19) United States
(12) Patent Application Publication
(10) Pub. No.: US 2023/0140478 A1
(43) Pub. Date: May 4, 2023

(54) Use of Lactoferrin

(71) Applicant: MJN U.S. Holdings LLC, Evansville, IN (US)

(72) Inventor: Zihua Ao, Evansville, IN (US)

(21) Appl. No.: 17/910,418

(22) PCT Filed: Mar. 16, 2021
(86) PCT No.: PCT/EP2021/056741

§ 371 Date: Sep. 9, 2022

(30) Foreign Application Priority Data
Mar. 16, 2020 (GB) 2003778.4

(51) Int. Cl. A61K 39/39; A61K 39/215

(52) U.S. Cl. CPC A61K 39/39; A61K 39/215; A61K 2039/55516

(57) ABSTRACT

The present application provides lactoferrin, and compositions comprising lactoferrin, for use in the treatment and/or prevention of the 2019 novel coronavirus (COVID-19) infection in a subject.

### FIELD OF THE INVENTION

The present application relates to a use of lactoferrin. In particular, the present application relates to lactoferrin, and compositions comprising lactoferrin, for the treatment and/or prevention of 2019 novel coronavirus (COVID-19; the “severe acute respiratory syndrome coronavirus 2 (SARS-CoV-2)”, as the novel coronavirus that causes COVID-19 infection has since been renamed) infection in a subject.

### BACKGROUND

In late 2019, an outbreak of pneumonia caused by the COVID-19 was reported in Wuhan City, China. As of 24 Feb. 2020, the COVID-19 virus had spread to 27 countries and had infected over 77,000 people in China alone, with the loss of over 2,500 lives.

Accordingly, there exists a need for novel ways of treating and/or preventing COVID-19 infection in a subject.

### SUMMARY OF INVENTION

In a first aspect, there is provided lactoferrin for use in the treatment and/or prevention of COVID-19 infection in a subject.

Preferably, the daily dosage of lactoferrin is in the range of about 1 mg/day to about 50 g/day.

Preferably, the lactoferrin is in the form of a reconstituted solution. More preferably, the reconstituted solution comprises lactoferrin in the range of about 0.1 mg/mL to about 0.25 g/mL.

Preferably, the lactoferrin is a COVID-19 immunologic adjuvant.

In a second aspect, there is provided lactoferrin for use as a COVID-19 immunologic adjuvant.

Preferably, the lactoferrin of the first aspect and/or second aspect is provided to the subject before, during, and/or after COVID-19 vaccine administration.

Preferably, the lactoferrin of the first aspect and/or second aspect is provided to the subject immediately prior to the COVID-19 vaccine administration, provided on the day of the COVID-19 vaccine administration but prior to COVID-19 vaccine administration, provided for at least one day prior to the COVID-19 vaccine administration, provided for at least two to four days prior to the COVID-19 vaccine administration, provided for at least five to seven days prior to the COVID-19 vaccine administration, provided for at least eight to fourteen days prior to the COVID-19 vaccine administration, provided for at least fifteen to twenty-nine days prior to the COVID-19 vaccine administration, provided for at least thirty to fifty-nine days prior to the COVID-19 vaccine administration, provided for at least sixty to eighty-nine days prior to the COVID-19 vaccine administration, or provided for at least ninety or more days prior to the COVID-19 vaccine administration.

Preferably, the lactoferrin of the first aspect and/or second aspect is provided to the subject immediately after the COVID-19 vaccine administration, provided on the day of the COVID-19 vaccine administration but after COVID-19 vaccine administration, provided for at least one day after the COVID-19 vaccine administration, provided for at least two to four days after the COVID-19 vaccine administration, provided for at least five to seven days after the COVID-19 vaccine administration, provided for at least eight to fourteen days after the COVID-19 vaccine administration, provided for at least fifteen to twenty-nine days after the COVID-19 vaccine administration, provided for at least thirty to fifty-nine days after the COVID-19 vaccine administration, provided for at least sixty to eighty-nine days after the COVID-19 vaccine administration, or provided for at least ninety or more days after the COVID-19 vaccine administration.

Preferably, the daily dosage of lactoferrin of the first aspect and/or second aspect is in the range of about 1 mg/day to about 50 g/day.

In a third aspect, there is provided a composition for use in the treatment and/or prevention of COVID-19 infection in a subject, wherein the composition comprises lactoferrin.

Preferably, the composition comprises lactoferrin in the range of about 15 mg/100 kcal to about 25 g/100 kcal.

Preferably, the composition is a nutritional composition.

Preferably, the composition is intended for a paediatric subject. Preferably, the lactoferrin is present in the range of about 15 mg/100 kcal to about 300 mg/100 kcal.

Alternatively, the composition is intended for an adult.

Preferably, the composition is a synthetic composition.

In a fourth aspect, there is provided a pharmaceutical composition for use in the treatment and/or prevention of COVID-19 infection in a subject, wherein the pharmaceutical composition comprises lactoferrin.

Preferably, the daily dosage of lactoferrin is in the range of about 1 mg/day to about 50 g/day.

Preferably, the pharmaceutical composition comprises at least one pharmaceutically acceptable excipient. More preferably, the at least one pharmaceutically acceptable excipient comprises a binder, a disintegrant, a diluent, a glidant, a solubilizer, a lubricant, a flavouring agent, any other pharmaceutically acceptable excipient, or a combination thereof.

Preferably, the pharmaceutical composition is a COVID-19 immunologic adjuvant.

In a fifth aspect, there is provided a pharmaceutical composition for use as a COVID-19 immunologic adjuvant, wherein the pharmaceutical composition comprises lactoferrin.

Preferably, the pharmaceutical composition of the fourth aspect and/or fifth aspect is provided to the subject before, during, and/or after COVID-19 vaccine administration.

Preferably, the pharmaceutical composition of the fourth aspect and/or fifth aspect is provided to the subject immediately prior to the COVID-19 vaccine administration, provided on the day of the COVID-19 vaccine administration but prior to COVID-19 vaccine administration, provided for at least one day prior to the COVID-19 vaccine administration, provided for at least two to four days prior to the COVID-19 vaccine administration, provided for at least five to seven days prior to the COVID-19 vaccine administration, provided for at least eight to fourteen days prior to the COVID-19 vaccine administration, provided for at least fifteen to twenty-nine days prior to the COVID-19 vaccine administration, provided for at least thirty to fifty-nine days prior to the COVID-19 vaccine administration, provided for at least sixty to eighty-nine days prior to the COVID-19 vaccine administration, or provided for at least ninety or more days prior to the COVID-19 vaccine administration.

Preferably, the pharmaceutical composition of the fourth aspect and/or fifth aspect is provided to the subject immediately after the COVID-19 vaccine administration, provided on the day of the COVID-19 vaccine administration but after COVID-19 vaccine administration, provided for at least one day after the COVID-19 vaccine administration, provided for at least two to four days after the COVID-19 vaccine administration, provided for at least five to seven days after the COVID-19 vaccine administration, provided for at least eight to fourteen days after the COVID-19 vaccine administration, provided for at least fifteen to twenty-nine days after the COVID-19 vaccine administration, provided for at least thirty to fifty-nine days after the COVID-19 vaccine administration, provided for at least sixty to eighty-nine days after the COVID-19 vaccine administration, or provided for at least ninety or more days after the COVID-19 vaccine administration.

Preferably, the daily dosage of lactoferrin of the fourth aspect and/or fifth aspect is in the range of about 1 mg/day to about 50 g/day.

In an aspect related to the first aspect and the second aspect, there is provided the use of lactoferrin in the manufacture of a composition for the treatment and/or prevention of 2019 novel coronavirus (COVID-19) infection in a subject.

In an aspect related to the third aspect, there is provided the use of a composition in the manufacture of a composition for the treatment and/or prevention of 2019 novel coronavirus (COVID-19) infection in a subject.

In an aspect related to the fourth aspect and the fifth aspect, there is provided the use of a pharmaceutical composition in the manufacture of a composition for the treatment and/or prevention of 2019 novel coronavirus (COVID-19) infection in a subject.

In an aspect related to the first aspect and the second aspect, there is provided a method for the treatment and/or prevention of 2019 novel coronavirus (COVID-19) infection in a subject, wherein the method comprises the step of administering lactoferrin to the subject.

In an aspect related to the third aspect, there is provided a method for the treatment and/or prevention of 2019 novel coronavirus (COVID-19) infection in a subject, wherein the method comprises the step of administering a composition to the subject, wherein the composition comprises lactoferrin.

In an aspect related to the fourth aspect and fifth aspect, there is provided a method for the treatment and/or prevention of 2019 novel coronavirus (COVID-19) infection in a subject, wherein the method comprises the step of administering a pharmaceutical composition to the subject, wherein the pharmaceutical composition comprises lactoferrin.

### Definitions

“Milk” means a substance that has been drawn or extracted from the mammary gland of a mammal.

“Milk-based composition” means a composition comprising any mammalian milk-derived or mammalian milk-based product known in the art. For example, a “milk-based composition” may comprise bovine casein, bovine whey, bovine lactose, or any combination thereof.

“Enriched milk product” generally refers to a milk ingredient that has been enriched with milk fat globule membrane (MFGM) and/or certain MFGM components, such as proteins and lipids found in the MFGM.

“Nutritional composition” means a substance or composition that satisfies at least a portion of a subject’s nutrient requirements. “Nutritional composition(s)” may refer to liquids, powders, solutions, gels, pastes, solids, concentrates, suspensions, ready-to-use forms of enteral formulas, oral formulas, formulas for infants, follow-up formulas, formulas for paediatric subjects, formulas for children, young child milks, and/or formulas for adults.

“Reconstituted solution”, in terms of the present disclosure, means the solution prepared when a diluent (e.g. water, saline, etc.) is added to an ingredient (e.g. a powder, a solution, a gel, a suspension, a paste, a solid, a liquid, a liquid concentrate, etc.). When the term “reconstituted solution” is used in reference to lactoferrin, this means the solution prepared by the addition of a diluent (e.g. water, saline, etc.) to a form of lactoferrin/lactoferrin-containing composition i.e. in the form of a powder, a solution, a gel, a suspension, a paste, a solid, a liquid, a liquid concentrate, etc.

The term “synthetic” when applied to a composition, a nutritional composition, or a mixture means a composition, nutritional composition, or mixture obtained by biological and/or chemical means, which can be chemically identical to the mixture naturally occurring in mammalian milks. A composition, nutritional composition, or mixture is said to be “synthetic” if at least one of its components is obtained by biological (e.g. enzymatic) and/or chemical means.

“Paediatric subject” means a human under 18 years of age. The term “adult”, in terms of the present disclosure, refers to a human that is 18 years of age or greater. The term “paediatric subject’ may refer to preterm infants, full-term infants, and/or children, as described below. A paediatric subject may be a human subject that is between birth and 8 years old. In another aspect, “paediatric subject” refers to a human subject between 1 and 6 years of age. Alternatively, “paediatric subject’ refers to a human subject between 6 and 12 years of age.

“Infant” means a human subject ranging in age from birth to not more than one year and includes infants from 0 to 12 months corrected age. The phrase “corrected age” means an infant’s chronological age minus the amount of time that the infant was born premature. Therefore, the corrected age is the age of the infant if it had been carried to full term. The term infant includes full-term infants, preterm infants, low birth weight infants, very low birth weight infants, and extremely low birth weight infants. “Preterm” means an infant born before the end of the 37^{th} week of gestation. “Full-term” means an infant born after the end of the 37^{th} week of gestation.

“Child’ means a subject ranging from 12 months to 13 years of age. A child may be a subject between the ages of 1 and 12 years old. In another aspect, the terms “children” or “child’ may refer to subjects that are between 1 and about 6 years old. Alternatively, the terms “children” or “child” may refer to subjects that are between about 7 and about 12 years old. The term “young child” means a subject ranging from 1 year to 3 years of age.

“Infant formula” means a composition that satisfies at least a portion of the nutrient requirements of an infant.

“Follow-up formula” means a composition that satisfies at least a portion of the nutrient requirements of an infant from the 6^{th} month onwards, and for young children from 1 to 3 years of age.

“Young child milk”, in terms of the present disclosure, means a fortified milk-based beverage intended for children over one year of age (typically from one to six years of age). Young child milks are designed with the intent to serve as a complement to a diverse diet, to provide additional insurance that a child achieves continual, daily intake of all essential vitamins and minerals, macronutrients plus additional functional dietary components, such as non-essential nutrients that have purported health-promoting properties.

The terms “adjuvant” and/or “immunologic adjuvant” are used interchangeably and refer to an ingredient that acts to accelerate, prolong, or enhance antigen-specific responses, in a subject, when used in combination with specific vaccine antigens. An immunologic adjuvant therefore increases and/or modulates a subject’s immune response to a vaccine. The enhancement of antigen-specific responses may be determined by the measurement of circulating blood biomarkers, such as receptor binding domain- (RBD-) binding immunoglobulin G (IgG) concentrations (U ml⁻¹) and/or geometric mean of SARS pseudovirus neutralisation titres (VNT50), using suitable bioanalytical methods known in the art.

The term “enteral” means deliverable through or within the gastrointestinal, or digestive, tract. “Enteral administration” includes oral feeding, intragastric feeding, transpyloric administration, or any other administration into the digestive tract. “Administration” is broader than “enteral administration” and includes parenteral administration or any other route of administration by which a substance is taken into a subject’s body.

The term “human milk oligosaccharides” or “HMOs” refers generally to a number of complex carbohydrates found in human breast milk.

The term “degree of hydrolysis” refers to the extent to which peptide bonds are broken by a hydrolysis method. The degree of protein hydrolysis for the purposes of characterising the hydrolysed protein component of the composition is easily determined by one of ordinary skill in the formulation arts, by quantifying the amino nitrogen to total nitrogen ratio (AN/TN) of the protein component of the selected composition. The amino nitrogen component is quantified by USP titration methods for determining amino nitrogen content, with the total nitrogen component being determined by the Kjeldahl method. These methods are well-known to one of ordinary skill in the analytical chemistry art.

The term “partially hydrolysed” means having a degree of hydrolysis which is greater than 0% but less than about 50%.

The term “extensively hydrolysed” means having a degree of hydrolysis which is greater than or equal to about 50%.

The term “peptide” describes linear molecular chains of amino acids, including single chain molecules or their fragments. The term “small amino acid peptide”, in terms of this disclosure, means a peptide comprising no more than 50 total amino acids. The small amino acid peptides of the present disclosure may be naturally occurring, or they may be synthesised.

The term “substantially free” means containing less than a functional amount of the specified component, typically less than 0.1% by weight, and includes 0% by weight of the specified ingredient.

As applied to nutrients, the term “essential” refers to any nutrient that cannot be synthesised by the body in amounts sufficient for normal growth, so it must be supplied by the diet. The term “conditionally essential” as applied to nutrients means that the nutrient must be supplied by the diet when adequate amounts of the precursor compound is unavailable to the body for endogenous synthesis to occur.

The term “probiotic” refers to microorganisms, such as bacteria or yeast, which have been shown to exert a beneficial effect on the health of a host subject. Probiotics can usually be classified as ‘viable’ or non-viable. The term ‘viable probiotics’ refers to living microorganisms, with the amount of a viable probiotic being detailed in colony-forming units (CFU). Probiotics that have been heat-killed, or otherwise inactivated, are termed ‘non-viable probiotics’ i.e. non-living microorganisms. Non-viable probiotics may still retain the ability to favourably influence the health of the host even though they may have been heat-killed or otherwise inactivated.

The term “prebiotic” refers to a non-digestible food ingredient that beneficially affects the host by selectively stimulating the growth and/or activity of one or a limited number of bacteria in the digestive tract, which can improve the health of the host. Prebiotics exert health benefits, which may include, but are not limited to: selective stimulation of the growth and/or activity of one or a limited number of beneficial gut bacteria; stimulation of the growth and/or activity of ingested probiotic microorganisms; selective reduction in gut pathogens; and, favourable influence on gut short chain fatty acid profile. The prebiotic of the composition may be naturally occurring, synthetic, or developed through the genetic manipulation of organisms and/or plants, whether such new source is now known or developed later.

The term “organism” refers to any contiguous living system, such as an animal, plant, fungus, or micro-organism.

“Non-human lactoferrin” refers to lactoferrin that is produced by or obtained from a source other than human breast milk.

The term “sialic acid” refers to a family of derivatives of neuraminic acid. N-acetylneuraminic acid (Neu5Ac) and N-glycolylneuraminic acid (Neu5Gc) are among the most abundant, naturally found forms of sialic acid, especially Neu5Ac in human and cow’s milk.

All percentages, parts, and ratios as used herein are detailed by weight of the total composition, unless otherwise specified. All amounts specified as administered “per day” may be delivered in a single unit dose, in a single serving, or in two or more doses or servings administered over the course of a 24-hour period.

All references to singular characteristics or limitations in the present disclosure shall include the corresponding plural characteristic or limitation, and vice versa, unless otherwise specified or clearly implied to the contrary, by the context in which the reference is made.

All combinations of method or process steps disclosed herein can be performed in any order, unless otherwise specified or clearly implied to the contrary, by the context in which the referenced combination is made.

The compositions of the present disclosure can comprise, consist of, or consist essentially of any of the components described herein, as well as including any additional useful component.

### DETAILED DESCRIPTION

COVID-19 (or the “severe acute respiratory syndrome coronavirus 2 (SARS-CoV-2)”, as the novel coronavirus that causes COVID-19 infection has since been renamed) is a previously unknown coronavirus. Zhu et al. (N Engl J Med 2020; 382: 727-733) and Lu et al. (The Lancet, 2020) determined that the sequence and genome structure of COVID-19 is highly homologous to that of the severe acute respiratory syndrome coronavirus (SARS-COV), which affected 26 countries and resulted in more than 8,000 cases between 2002 and 2004.

The SARS-COV is known to enter cells via an interaction with its functional receptor, the angiotensin-converting enzyme 2 (ACE-2; Liang et al., medRxiv, 2020). COVID-19 has also been shown to utilise the ACE-2 cell entry receptor (Wan et al., Journal of Virology, 2020). As Lang et al. (PLoS One, August 2011, 6(8)) demonstrated that lactoferrin is able to prevent SARS-COV pseudovirus cell entry, it is hypothesised that lactoferrin will be useful in the treatment and/or prevention of COVID-19 infection in a subject.

In addition, it is known that lactoferrin is able to bridge and co-ordinate signalling between the innate and adaptive immune systems. Lactoferrin is secreted by macrophages in response to inflammation, as well as other stimuli (e.g. adjuncts, such as aluminium salts), with an improved influenza H1N1 vaccine response demonstrated when concurrently administered with bovine lactoferrin (Sherman et al., Biochem. Biophys. Res. Commun., 2015). It is therefore hypothesised that lactoferrin, as well as compositions comprising lactoferrin, will act as an effective immunologic adjuvant during COVID-19 vaccination in a subject, by supplementing or increasing the circulating volume of lactoferrin in the subject.

The present invention therefore provides lactoferrin for use in the treatment and/or prevention of COVID-19 infection in a subject, as well as providing novel compositions comprising lactoferrin for the treatment and/or prevention of COVID-19 infection in a subject.

The lactoferrin of any aspect detailed below may comprise human lactoferrin produced by a genetically modified organism, humanised lactoferrin, non-human lactoferrin, or any combination thereof. The non-human lactoferrin may comprise bovine lactoferrin, porcine lactoferrin, equine lactoferrin, buffalo lactoferrin, goat lactoferrin, murine lactoferrin, or camel lactoferrin

In one aspect, the present invention provides lactoferrin for use in the treatment and/or prevention of COVID-19 infection in a subject. The lactoferrin may be administered to the subject in the form of a powder, a solution, a gel, a suspension, a paste, a solid, a liquid, a liquid concentrate, or a reconstituted solution. Preferably, the lactoferrin is administered to the subject in the form of a powder, a liquid concentrate, or a reconstituted solution. More preferably, the lactoferrin is administered to the subject in the form of a reconstituted solution.

The lactoferrin daily dosage may be varied depending on the requirement of the patient, the severity of the infection, and the particular form of lactoferrin. The daily dosage of lactoferrin may be in the range of about 1 milligram per day (mg/day) to about 50 grams per day (g/day). Preferably, the daily dosage of lactoferrin is in the range of about 1 mg/day to about 10 g/day. More preferably, the daily dosage of lactoferrin is in the range of about 1 mg/day to about 5 g/day. Even more preferably, the daily dosage of lactoferrin is in the range of about 5 mg/day to about 2 g/day. The dose of lactoferrin may be in the form of a single daily dosage. Alternatively, the total daily dosage may be administered in portions throughout the day e.g. two portions, three portions, etc.

The lactoferrin may be administered to the subject in an amount suitable to treat and/or prevent COVID-19 infection in a subject. The lactoferrin may be administered as a reconstituted solution comprising lactoferrin in the range of about 0.01 milligrams per millilitre (mg/mL) to about 0.25 grams per millilitre (g/mL). Preferably, the reconstituted solution comprises lactoferrin in the range of about 0.1 mg/mL to about 200 mg/mL. More preferably, the reconstituted solution comprises lactoferrin in the range of about 0.1 mg/mL to about 100 mg/mL.

Alternatively, the lactoferrin may be administered as a reconstituted solution comprising lactoferrin in the range of about 15 milligrams per 100 kilocalories (mg/100 kcal) to about 25 grams per 100 kilocalories (g/100 kcal). Preferably, the reconstituted solution comprises lactoferrin in the range of about 5 g/100 kcal to about 20 g/100 kcal. More preferably, the reconstituted solution comprises lactoferrin in the range of about 10 g/100 kcal to about 15 g/100 kcal.

Alternatively, the lactoferrin may be administered as a reconstituted solution comprising lactoferrin in an amount of about 0.01% weight per volume (% w/v) to about 25% w/v. Preferably, the reconstituted solution comprises lactoferrin in the range of about 0.1% w/v to about 20% w/v. More preferably, the reconstituted solution comprises lactoferrin in the range of about 0.1% w/v to about 10% w/v.

The lactoferrin may be a COVID-19 immunologic adjuvant. The lactoferrin may be for use as a COVID-19 immunologic adjuvant. The lactoferrin may be provided to the subject before, during, and/or after COVID-19 vaccine administration. The lactoferrin may therefore be provided to the subject before COVID-19 vaccine administration, at the same time as the COVID-19 vaccine administration (i.e. in the same composition as the COVID-19 vaccine), and/or after COVID-19 vaccine administration.

The lactoferrin may be provided to the subject immediately prior to COVID-19 vaccine administration, on the day of the COVID-19 vaccine administration but prior to COVID-19 vaccine administration, for at least one day prior to the COVID-19 vaccine administration, for at least two to four days prior to the COVID-19 vaccine administration, for at least five to seven days prior to the COVID-19 vaccine administration, for at least eight to fourteen days prior to the COVID-19 vaccine administration, for at least fifteen to twenty-nine days prior to the COVID-19 vaccine administration, for at least thirty to fifty-nine days prior to the COVID-19 vaccine administration, for at least sixty to eighty-nine days prior to the COVID-19 vaccine administration, or for at least ninety or more days prior to the COVID-19 vaccine administration.

The lactoferrin may be provided to the subject immediately after COVID-19 vaccine administration, on the day of the COVID-19 vaccine administration but after COVID-19 vaccine administration, for at least one day after the COVID-19 vaccine administration, for at least two to four days after the COVID-19 vaccine administration, for at least five to seven days after the COVID-19 vaccine administration, for at least eight to fourteen days after the COVID-19 vaccine administration, for at least fifteen to twenty-nine days after the COVID-19 vaccine administration, for at least thirty to fifty-nine days after the COVID-19 vaccine administration, for at least sixty to eighty-nine days after the COVID-19 vaccine administration, or for at least ninety or more days after the COVID-19 vaccine administration.

The lactoferrin may be provided to the subject before the COVID-19 vaccine administration, during COVID-19 vaccine administration, and after the COVID-19 vaccine administration, in any combination previously discussed.

When the lactoferrin is a COVID-19 immunologic adjuvant, the daily dosage of lactoferrin may be in the range of about 1 mg/day to about 50 g/day. Preferably, the daily dosage of lactoferrin is in the range of about 50 mg/day to about 2000 mg/day. More preferably, the daily dosage of lactoferrin is in the range of about 200 mg/day to about 1600 mg/day. Even more preferably, the daily dosage of lactoferrin is in the range of about 300 mg/day to about 1200 mg/day. Yet even more preferably, the daily dosage of lactoferrin is in the range of about 400 mg/day to about 1000 mg/day. The dose of lactoferrin may be in the form of a single daily dosage. Alternatively, the total daily dosage may be administered in portions throughout the day e.g. two portions, three portions, etc.

In another aspect, the present invention provides a composition for the treatment and/or prevention of COVID-19 infection in a subject, wherein the composition comprises lactoferrin.

Lactoferrin may be present in the composition in an amount of at least about 15 mg/100 kcal. The composition may comprise lactoferrin in the range of about 15 mg/100 kcal to about 25 g/100 kcal. Preferably, the composition comprises lactoferrin in the range of about 5 g/100 kcal to about 20 g/100 kcal. More preferably, the composition comprises lactoferrin in the range of about 10 g/100 kcal to about 15 g/100 kcal. The composition may be specifically designed for a paediatric subject. When the composition is specifically intended for a paediatric subject, the composition may comprise lactoferrin in the range of about 15 mg/100 kcal to about 300 mg/100 kcal. Preferably, the composition comprises lactoferrin in the range of about 60 mg to about 150 mg/100 kcal. More preferably, the composition comprises lactoferrin in the range of about 60 mg/100 kcal to about 100 mg/100 kcal.

The composition may be provided in any form known in the art. The composition may be provided in the form of a powder, a gel, a suspension, a paste, a solid, a liquid, a liquid concentrate, a reconstitutable powder, a reconstituted solution, or a ready-to-use product. Preferably, the composition is in the form of a reconstitutable powder, a reconstituted solution, or a ready-to-use product. Most preferably, the composition is provided in the form of a reconstitutable powder.

When the composition comprises a reconstituted solution, the reconstituted solution may comprise lactoferrin in the range of about 0.5 mg/mL to about 1.5 mg/mL. Preferably, the reconstituted solution comprises lactoferrin in the range of about 0.6 mg/mL to about 1.3 mg/mL. Alternatively, the reconstituted solution may comprise lactoferrin in the range of about 0.1 grams per litre (g/L) to about 2 g/L. Preferably, the reconstituted solution comprises lactoferrin in the range of about 0.6 g/L to about 1.5 g/L.

The composition may comprise a protein source, a fat or lipid source, a carbohydrate source, or any combination thereof. The composition may comprise one or more: probiotics; prebiotics; source of long chain polyunsaturated fatty acids (LCPUFAs); human milk oligosaccharides (HMOs); β-glucan; sialic acid; suitable composition ingredient; or, any combination thereof.

The composition may comprise at least one protein source, in addition to lactoferrin, wherein the protein source provides further protein to the composition. The protein source may comprise intact protein, partially hydrolysed protein, extensively hydrolysed protein, small amino acid peptides, or any combination thereof. The protein source may be derived from any mammalian animal milk protein or plant protein, as well as their fractions, or any combination thereof. The protein source may comprise bovine milk, caprine milk, whey protein, casein protein, soy protein, rice protein, pea protein, peanut protein, egg protein, sesame protein, fish protein, wheat protein, hydrolysed protein, or any combination thereof. Bovine milk protein sources may comprise, but are not limited to, milk protein powders, milk protein concentrates, milk protein isolates, non-fat milk solids, non-fat milk, non-fat dry milk, whey protein, whey protein isolates, whey protein concentrates, sweet whey, acid whey, casein, acid casein, caseinate (e.g. sodium caseinate, sodium calcium caseinate, calcium caseinate), or any combination thereof.

The composition may comprise a protein source in the range of about 1 g/100 kcal to about 7 g/100 kcal. Preferably, the composition comprises a protein source in the range of about 3.5 g/100 kcal to about 4.5 g/100 kcal. The protein source may comprise from about 40% to about 85% whey protein and from about 15% to about 60% casein.

As noted above, the protein source may comprise a source of intact protein. The composition may comprise intact protein in the range of about 1 g/100 kcal to about 3 g/100 kcal. Preferably, the composition comprises intact protein in the range of about 1 g/100 kcal to about 2.5 g/100 kcal. More preferably, the composition comprises intact protein in the range of about 1.3 g/100 kcal to about 2.1 g/100 kcal. The protein source may comprise a combination of intact protein and partially hydrolysed protein, wherein the partially hydrolysed protein may have a degree of hydrolysis of between about 4% and 10%.

As also noted above, the protein source of the composition may comprise partially hydrolysed protein, extensively hydrolysed protein, or a combination thereof. The hydrolysed proteins may be treated with enzymes to break down some or most of the proteins that cause adverse symptoms with the goal of reducing allergic reactions, intolerance, and sensitisation. The proteins may be hydrolysed by any method known in the art. The terms “protein hydrolysates” or “hydrolysed protein” are used interchangeably herein and refer to hydrolysed proteins, wherein the degree of hydrolysis may be from about 20% to about 80%, or from about 30% to about 80%, or even from about 40% to about 60%.

The composition may be substantially free of protein and may comprise free amino acids as a protein equivalent source. The amino acids may comprise histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine, or any combination thereof. The amino acids may be branched chain amino acids. The amount of free amino acids in the composition may vary from about 1 to about 5 g/100 kcal. The free amino acids may all have a molecular weight of less than 500 Da. As the composition may be substantially free of protein and thus, devoid of the proteins that cause adverse symptoms, the composition may be hypoallergenic.

The composition may comprise an enriched milk product. The enriched milk product may be formed by fractionation of non-human (e.g. bovine) milk. The enriched milk product may have a total protein level in a range of between 20% and 90%; preferably, the enriched milk product has a total protein level in a range of between 65% and 80%.

The enriched milk product may comprise an enriched whey protein concentrate (eWPC). Alternatively, the enriched milk product may comprise an enriched lipid fraction derived from milk. The eWPC and the enriched lipid fraction may be produced by any number of fractionation techniques well known in the art. These techniques include, but are not limited to, membrane filtration, melting point fractionation, organic solvent fractionation, super critical fluid fractionation, or any combination thereof. Alternatively, eWPC is available commercially, including under the trade names Lacprodan MFGM-10 and Lacprodan PL-20, both available from Arla Food Ingredients of Viby, Denmark. With the addition of eWPC, the lipid composition of the composition can more closely resemble that of human milk.

The composition may comprise eWPC in the range of about 0.5 g/L to about 10 g/L. Preferably, the composition comprises eWPC in the range of about 1 g/L to about 9 g/L. More preferably, the composition comprises eWPC in the range of about 3 g/L to about 8 g/L. Alternatively, the composition may comprise eWPC in the range of about 0.06 g/100 kcal to about 1.5 g/100 kcal. Preferably, the composition comprises eWPC in the range of about 0.3 g/100 kcal to about 1.4 g/100 kcal. More preferably, the composition comprises eWPC in the range of about 0.4 g/100 kcal to about 1 g/100 kcal.

The composition may comprise at least one fat or lipid source, wherein the fat or lipid source provides fat and/or lipid to the composition. Suitable fat or lipid sources for the composition may be any known or used in the art. The fat or lipid source may be present in the composition in addition to another fat or lipid source, such as a LCPUFA. The fat or lipid source may comprise animal sources, such as milk fat, butter, butter fat, or egg yolk lipid; marine sources, such as fish oils, marine oils, or single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, or wheat germ oil; medium chain triglyceride oils; emulsions and esters of fatty acids; or any combination thereof.

The composition may comprise a fat or lipid source in the range of about 1 g/100 kcal to about 10 g/100 kcal. Preferably, the composition comprises a fat or lipid source in the range of about 2 g/100 kcal to about 7 g/100 kcal of a fat or lipid source. More preferably the composition comprises a fat or lipid source in the range of about 2.5 g/100 kcal to about 6 g/100 kcal. Most preferably, the composition comprises a fat or lipid source in the range of about 3 g/100 kcal to about 4 g/100 kcal.

The composition may comprise at least one carbohydrate source, wherein the carbohydrate source provides carbohydrate to the composition. The carbohydrate source may be present in the composition in addition to another carbohydrate source, such as PDX and GOS. The carbohydrate source may comprise lactose, glucose, fructose, maltodextrins, sucrose, starch, maltodextrin, maltose, fructooligosaccharides, corn syrup, high fructose corn syrup, dextrose, corn syrup solids, rice syrup solids, or any combination thereof. Moreover, hydrolysed, partially hydrolysed, and/or extensively hydrolysed carbohydrates may be desirable for inclusion in the composition due to their easy digestibility. More specifically, hydrolysed carbohydrates are less likely to contain allergenic epitopes. The composition may therefore comprise a carbohydrate source comprising hydrolysed or intact, naturally or chemically modified, starches sourced from corn, tapioca, rice, or potato, in waxy or non-waxy forms, such as hydrolysed corn starch.

The composition may comprise a carbohydrate source in the range of about 5 g/100 kcal to about 25 g/100 kcal. Preferably, the composition comprises a carbohydrate source in the range of about 6 g/100 kcal to about 22 g/100 kcal. More preferably, the composition comprises a carbohydrate source in the range of about 12 g/100 kcal to about 14 g/100 kcal.

The composition may comprise one or more probiotics. The probiotic may comprise any Bifidobacterium species, any Lactobacillus species, or a combination thereof. Preferably, the probiotic is Bifidobacterium adolescentis (ATCC number 15703), Bifidobacterium animalis subsp. lactis, Bifidobacterium breve, Bifidobacterium longum subsp. infantis (B. infantis), Lactobacillus acidophilus, Lactobacillus gasseri (ATCC number 33323), Lactobacillus reuteri (DSM number 17938), Lactobacillus rhamnosus GG (LGG; ATCC number 53103), or any combination thereof. More preferably, the probiotic is LGG, B. infantis, or a combination thereof.

The probiotic may be viable or non-viable. The probiotic incorporated into the composition may comprise both viable colony-forming units and non-viable probiotic cell-equivalents. The probiotic may be naturally occurring, synthetic, or developed through the genetic manipulation of organisms, whether such source is now known or later developed.

The composition may comprise a viable probiotic in the range of about 1 x 10⁴ colony forming units per 100 kilocalories (CFU/100 kcal) to about 1.5 x 10¹² CFU/100 kcal. Preferably, the composition comprises a viable probiotic in the range of about 1 x 10⁶ CFU/100 kcal to about 1 x 10⁹ CFU/100 kcal. More preferably, the composition comprises a viable probiotic in the range of about 1 x 10⁷ CFU/100 kcal to about 1 x 10⁸ CFU/100 kcal.

The composition may comprise one or more prebiotics. The prebiotic may comprise oligosaccharides, polysaccharides, or any other prebiotics that comprise fructose, xylose, soya, galactose, glucose, mannose, or any combination thereof. More specifically, the prebiotic may comprise polydextrose (PDX), polydextrose powder, lactulose, lactosucrose, raffinose, glucooligosaccharides, inulin, fructooligosaccharides, isomaltooligosaccharides, soybean oligosaccharides, lactosucrose, xylooligosaccharides, chitooligosaccharides, mannooligosaccharides, aribino-oligosaccharides, sialyloligosaccharides, fucooligosaccharides, galactooligosaccharides (GOS), and gentiooligosaccharides.

The composition may comprise a prebiotic in the range of about 1.0 g/L to about 10.0 g/L of the composition. Preferably, the composition comprises a prebiotic in the range of about 2.0 g/L and about 8.0 g/L of the composition. Alternatively, the composition may comprise a prebiotic in the range of about 0.01 g/100 kcal to about 1.5 g/100 kcal. Preferably, the composition comprises a prebiotic in the range of about 0.15 g/100 kcal to about 1.5 g/100 kcal.

The composition may comprise a prebiotic comprising PDX, GOS, or a combination thereof.

The composition may comprise PDX in the range of about 1.0 g/L and 10.0 g/L. Preferably, the composition comprises PDX in the range of about 2.0 g/L and 8.0 g/L. Alternatively, the composition comprises PDX in the range of about 0.015 g/100 kcal to about 1.5 g/100 kcal. Preferably, the composition comprises PDX in the range of about 0.05 g/100 kcal to about 1.5 g/100 kcal. More preferably, the composition comprises PDX in the range of about 0.2 g/100 kcal to about 0.6 g/100 kcal.

The composition may comprise GOS in the range of about 0.015 g/100 kcal to about 1.0 g/100 kcal. Preferably, the composition comprises GOS in the range of about 0.2 g/100 kcal to about 0.5 g/100 kcal.

The composition may comprise PDX in combination with GOS. Advantageously, the combination of PDX and GOS may stimulate and/or enhance endogenous butyrate production by microbiota. The composition may comprise GOS and PDX in a total amount of at least about 0.015 g/100 kcal. The composition may comprise GOS and PDX in a total amount in the range of about 0.015 g/100 kcal to about 1.5 g/100 kcal. Preferably, the composition comprises GOS and PDX in a total amount in the range of about 0.1 g/100 kcal to about 1.0 g/100 kcal. The prebiotic may comprise at least 20% weight per weight (w/w) PDX, GOS, or a combination thereof.

The composition may comprise one or more human milk oligosaccharides (HMOs). The HMO may comprise 2′-fucosyllactose (2FL), 3′-fucosyllactose (3FL), lacto-N-tetraose (LNT), lacto-N-neotetraose (LNnT), lacto-N-fucopentaose I (LNFP-I), 3′-sialyllactose (3SL), 6′-sialyllactose (6SL), or a combination thereof.

The composition may comprise an HMO in the range of about 0.01 g/L to about 5.0 g/L. Preferably, the composition comprises an HMO in the range of about 0.05 g/L to about 4.0 g/L of the composition. More preferably, the composition comprises an HMO in the range of about 0.05 g/L to about 2.0 g/L of the composition. Alternatively, the composition may comprise an HMO in the range of about 0.01 g/100 kcal to about 2.0 g/100 kcal. Preferably, the composition comprises an HMO in the range of about 0.01 g/100 kcal to about 1.5 g/100 kcal.

The composition may comprise a source of long-chain polyunsaturated fatty acids (LCPUFAs). The source of LCPUFAs may comprise docosahexaenoic acid (DHA), α-linoleic acid, γ-linoleic acid, linoleic acid, linolenic acid, eicosapentaenoic acid (EPA), arachidonic acid (ARA), or any combination thereof. Preferably, the composition comprises a source of LCPUFAs comprising DHA, ARA, or a combination thereof.

The composition may comprise an LCPUFA in an amount of at least about 5 mg/100 kcal. The composition may comprise an LCPUFA in the range of about 5 mg/100 kcal to about 100 mg/100 kcal. Preferably, the composition comprises an LCPUFA in the range of about 10 mg/100 kcal to about 50 mg/100 kcal.

The composition may comprise DHA in the range of about 5 mg/100 kcal to about 80 mg/100 kcal. Preferably, the composition comprises DHA in the range of about 10 mg/100 kcal to about 20 mg/100 kcal. More preferably, the composition comprises DHA in the range of about 15 mg/100 kcal to about 20 mg/100 kcal.

The composition may comprise ARA in the range of about 10 mg/100 kcal to about 100 mg/100 kcal of ARA. Preferably, the composition comprises ARA in the range of about 15 mg/100 kcal to about 70 mg/100 kcal. More preferably, the composition comprises ARA in the range of about 20 mg/100 kcal to about 40 mg/100 kcal.

The composition may comprise both DHA and ARA. The weight ratio of ARA:DHA may be in the range of about 1:3 to about 9:1. Preferably, the weight ratio of ARA:DHA is in the range of about 1:2 to about 4:1. The composition may comprise oils containing DHA and/or ARA. If utilised, the source of DHA and/or ARA may be any source known in the art such as marine oil, fish oil, single cell oil, egg yolk lipid, or brain lipid. The DHA and ARA may be sourced from single cell oils, DHASCO® and ARASCO® from DSM Nutritional Products, or variations thereof. The DHA and ARA may be in a natural form, provided that the remainder of the LCPUFA source does not result in any substantial deleterious effect on the subject. Alternatively, the DHA and ARA may be used in refined form.

The composition may comprise β-glucan. Preferably, the β-glucan comprises β-1,3-glucan. Preferably, the β-1,3-glucan comprises β-1,3;1,6-glucan. The composition may comprise β-glucan present in the range of about 0.010 grams to about 0.080 grams per 100 g of composition. Alternatively, the composition may comprise β-glucan in the range of about 3 mg/100 kcal to about 17 mg/100 kcal. Preferably, the composition comprises β-glucan in the range of about 4 mg/100 kcal to about 17 mg/100 kcal.

The composition may comprise sialic acid. Mammalian brain tissue contains the highest levels of sialic acid as sialic acid is incorporated into brain-specific proteins, such as the neural cell adhesion molecule (NCAM) and lipids (e.g. gangliosides). Sialic acid is therefore believed to play an important role in neural development and function, learning, cognition, and memory.

The composition may comprise sialic acid provided by an inherent source (such as eWPC), exogenous sialic acid, sialic acid from sources (such as cGMP), or any combination thereof. The composition may comprise sialic acid in the range of about 100 mg/L to about 800 mg/L. Preferably, the composition comprises sialic acid in the range of about 120 mg/L to about 600 mg/L. More preferably, the composition comprises sialic acid in the range of about 140 mg/L to about 500 mg/L. Alternatively, the composition may comprise sialic acid in the range of about 1 mg/100 kcal to about 120 mg/100 kcal. Preferably, the composition comprises sialic acid in the range of about 14 mg/100 kcal to about 90 mg/100 kcal. More preferably, the composition comprises sialic acid in the range of about 15 mg/100 kcal to about 75 mg/100 kcal.

The composition may comprise one or more suitable composition ingredient, wherein the suitable composition ingredient comprises folic acid, arginine, choline, inositol, an emulsifier, a preservative, a stabiliser, or a combination thereof. The composition may comprise choline. Choline is a nutrient that is essential for normal function of cells. Choline is a precursor for membrane phospholipids and it accelerates the synthesis and release of acetylcholine, a neurotransmitter involved in memory storage. Without wishing to be bound by theory, it is believed that dietary choline and docosahexaenoic acid (DHA) act synergistically to promote the biosynthesis of phosphatidylcholine and thus, help promote synaptogenesis in human subjects. Additionally, choline and DHA act synergistically to promote dendritic spine formation, which is important in the maintenance of established synaptic connections. The composition may comprise about 20 mg to about 100 mg of choline per 8 fl. oz. (236.6 mL) serving.

The composition may comprise inositol. The inositol may be present as exogenous inositol, inherent inositol, or a combination thereof. The composition may comprise inositol in the range of about 10 mg/100 kcal to 40 mg/100 kcal. Preferably, the composition comprises inositol in the range of about 20 mg/100 kcal to 40 mg/100 kcal. Alternatively, the composition comprises inositol in the range of about 130 mg/L to about 300 mg/L.

The composition may comprise one or more emulsifier, as an emulsifier can increase the stability of the composition. The emulsifier may comprise, but is not limited to, egg lecithin, soy lecithin, alpha lactalbumin, monoglycerides, diglycerides, or any combination thereof.

The composition may comprise one or more preservative, as a preservative can extend the shelf-life of the composition. The preservative may comprise, but is not limited to, potassium sorbate, sodium sorbate, potassium benzoate, sodium benzoate, calcium disodium EDTA, or any combination thereof.

The composition may comprise one or more stabiliser, as a stabiliser can help preserve the structure of the composition. The stabiliser may comprise, but is not limited to, gum arabic, gum ghatti, gum karaya, gum tragacanth, agar, furcellaran, guar gum, gellan gum, locust bean gum, pectin, low methoxyl pectin, gelatine, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, or any combination thereof.

The composition may be intended for a paediatric subject or an adult. The paediatric subject may be an infant or a child. The infant may be a vaginally-delivered infant. Alternatively, the infant may be an infant delivered by C-section. The gut microbiota play a significant role in the development and maturation of the immune system. It is known that the gut microbiota of C-section infants is different to infants that were vaginally delivered, with a study showing that C-section birth is associated with an increased likelihood of immune and metabolic disorders such as allergies, asthma, hypertension, and obesity (Hansen et al., J Immunol Aug. 1, 2014, 193 (3) 1213-1222). One possible way of reducing the likelihood of immune and metabolic disorders in C-section infants may be the provision of a composition comprising lactoferrin and beneficial probiotics such as LGG and B. infantis, in an attempt to bring the gut microbiota of the C-section infants into closer alignment with the gut microbiota of vaginally-delivered infants.

The composition may be provided in a form suitable for enteral administration, topical administration, and/or parenteral administration. The composition may therefore be provided in a form suitable for oral administration, topical administration, subcutaneous administration, intravenous administration, intramuscular administration, intradermal administration, nasal administration, intranasal administration, rectal administration, ocular administration, vaginal administration, and/or any other commonly known form of administration.

When the composition is provided in a form suitable for enteral administration, the composition may comprise a nutritional supplement, an adult’s nutritional product, a children’s nutritional product, an infant formula, a human milk fortifier, a follow-up formula, a young child milk, or any other composition designed for an infant or a paediatric subject. The composition may be provided in an orally-ingestible form, wherein the orally-ingestible comprises a food, a beverage, a tablet, a capsule, or a powder.

The composition may be expelled directly into a subject’s intestinal tract. The composition may be expelled directly into the gut. The composition may be formulated to be consumed or administered enterally under the supervision of a physician. The composition may be formulated to be administered parenterally.

The composition may be suitable for a number of dietary requirements. The composition may be kosher. The composition may be a non-genetically modified product. The composition may be sucrose-free. The composition may also be lactose-free. The composition may not contain any medium-chain triglyceride oil. No carrageenan may be present in the composition. The composition may be free of all gums.

The composition may be provided in a form suitable for topical administration and/or parenteral administration. The composition may comprise a patch, a microneedle patch, a transdermal patch, a gel, a hydrogel, a cream, a solution, an ointment, a balm, a spray, a suspension, a paste, a reconstituted solution, or any combination thereof.

In yet another aspect, the present invention provides a pharmaceutical composition for the treatment and/or prevention of COVID-19 infection in a subject, wherein the pharmaceutical composition comprises lactoferrin.

The pharmaceutical composition may be provided in any form known in the art. The pharmaceutical composition may be provided in a form suitable for enteral administration, topical administration, and/or parenteral administration. The pharmaceutical composition may therefore be provided in a form suitable for oral administration, topical administration, subcutaneous administration, intravenous administration, intramuscular administration, intradermal administration, nasal administration, intranasal administration, and/or any other commonly known form of administration.

The pharmaceutical composition may be provided in a solid form preparation or a fluid preparation. When the pharmaceutical composition is a solid form preparation, the pharmaceutical composition may be provided as a tablet, a capsule, a reconstitutable powder, or as dispersible granules. When the pharmaceutical composition is a fluid preparation, the pharmaceutical composition may be provided in the form of a gel, a hydrogel, a suspension, a paste, a solution, a reconstituted solution, a patch, a microneedle patch, a transdermal patch, a cream, an ointment, a balm, a spray, or any combination thereof. Preferably, the pharmaceutical composition is in the form of a tablet, a capsule, a reconstitutable powder, or a reconstituted solution. Most preferably, the pharmaceutical composition is provided in the form of a tablet.

The lactoferrin daily dosage may be varied depending on the requirement of the patient, the severity of the infection, and the particular form of lactoferrin. The daily dosage of lactoferrin may be in the range of about 1 mg/day to about 50 g/day. Preferably, the daily dosage of lactoferrin is in the range of about 1 mg/day to about 10 g/day. More preferably, the daily dosage of lactoferrin is in the range of about 1 mg/day to about 5 g/day. Even more preferably, the daily dosage of lactoferrin is in the range of about 5 mg/day to about 2 g/day. The dose of lactoferrin may be in the form of a single daily dosage. Alternatively, the total daily dosage may be administered in portions throughout the day e.g. two portions, three portions, etc.

The lactoferrin may be administered to the subject in an amount suitable to treat and/or prevent COVID-19 infection in a subject. The lactoferrin may be administered as a solid form or fluid preparation comprising lactoferrin in the range of about 0.01 mg/mL to about 0.25 g/mL. Preferably, the solid form or fluid preparation comprises lactoferrin in the range of about 0.1 mg/mL to about 200 mg/mL. More preferably, the solid form or fluid preparation comprises lactoferrin in the range of about 0.1 mg/mL to about 100 mg/mL.

Alternatively, the lactoferrin may be administered as a solid form or fluid preparation comprising lactoferrin in the range of about 15 mg/100 kcal to about 25 g/100 kcal. Preferably, the solid form or fluid preparation comprises lactoferrin in the range of about 5 g/100 kcal to about 20 g/100 kcal. More preferably, the solid form or fluid preparation comprises lactoferrin in the range of about 10 g/100 kcal to about 15 g/100 kcal.

The pharmaceutical composition may comprise at least one pharmaceutically acceptable excipient. The pharmaceutically acceptable excipient may comprise a binder, a disintegrant, a diluent, a glidant, a solubilizer, a lubricant, a flavouring agent, any other pharmaceutically acceptable excipient, or a combination thereof.

The pharmaceutical composition may be a COVID-19 immunologic adjuvant. The pharmaceutical composition may be for use as a COVID-19 immunologic adjuvant. The pharmaceutical composition may be provided to the subject before, during, and/or after COVID-19 vaccine administration. The pharmaceutical composition may therefore be provided to the subject before COVID-19 vaccine administration, at the same time as the COVID-19 vaccine administration (i.e. in the same composition as the COVID-19 vaccine), and/or after COVID-19 vaccine administration.

The pharmaceutical composition may be provided to the subject immediately prior to COVID-19 vaccine administration, on the day of the COVID-19 vaccine administration but prior to COVID-19 vaccine administration, for at least one day prior to the COVID-19 vaccine administration, for at least two to four days prior to the COVID-19 vaccine administration, for at least five to seven days prior to the COVID-19 vaccine administration, for at least eight to fourteen days prior to the COVID-19 vaccine administration, for at least fifteen to twenty-nine days prior to the COVID-19 vaccine administration, for at least thirty to fifty-nine days prior to the COVID-19 vaccine administration, for at least sixty to eighty-nine days prior to the COVID-19 vaccine administration, or for at least ninety or more days prior to the COVID-19 vaccine administration.

The pharmaceutical composition may be provided to the subject immediately after COVID-19 vaccine administration, on the day of the COVID-19 vaccine administration but after COVID-19 vaccine administration, for at least one day after the COVID-19 vaccine administration, for at least two to four days after the COVID-19 vaccine administration, for at least five to seven days after the COVID-19 vaccine administration, for at least eight to fourteen days after the COVID-19 vaccine administration, for at least fifteen to twenty-nine days after the COVID-19 vaccine administration, for at least thirty to fifty-nine days after the COVID-19 vaccine administration, for at least sixty to eighty-nine days after the COVID-19 vaccine administration, or for at least ninety or more days after the COVID-19 vaccine administration.

The pharmaceutical composition may be provided to the subject before the COVID-19 vaccine administration, during COVID-19 vaccine administration, and after the COVID-19 vaccine administration, in any combination previously discussed.

When the pharmaceutical composition is a COVID-19 immunologic adjuvant, the daily dosage of lactoferrin may be in the range of about 1 mg/day to about 50 g/day. Preferably, the daily dosage of lactoferrin is in the range of about 50 mg/day to about 2000 mg/day. More preferably, the daily dosage of lactoferrin is in the range of about 200 mg/day to about 1600 mg/day. Even more preferably, the daily dosage of lactoferrin is in the range of about 300 mg/day to about 1200 mg/day. Yet even more preferably, the daily dosage of lactoferrin is in the range of about 400 mg/day to about 1000 mg/day. The dose of lactoferrin may be in the form of a single daily dosage. Alternatively, the total daily dosage may be administered in portions throughout the day e.g. two portions, three portions, etc.

The scope of the present invention is defined in the appended claims. It is to be understood that the skilled person may make amendments to the scope of the claims without departing from the scope of the present disclosure.

### Example Compositions

The compositions shown in Table 1 illustrate examples of compositions within the scope of the present disclosure, but are in no way intended to provide any limitation on the disclosure.

TABLE 1

| Component | Composition | | | | | | | | | | | | |
|---|---|---|---|---|---|---|---|---|---|---|---|---|---|
| | I | II | III | IV | V | VI | VII | VIII | IX | X | XI | XII | XIII |
| Lactoferrin | ✔ | ✔ | ✔ | ✔ | ✔ | ✔ | ✔ | ✔ | ✔ | ✔ | ✔ | ✔ | ✔ |
| LGG | X | ✔ | X | X | X | X | X | ✔ | ✔ | X | X | X | ✔ |
| B. infantis | X | X | X | X | X | X | X | X | X | ✔ | ✔ | ✔ | ✔ |
| Enriched milk product | X | X | ✔ | X | X | X | X | ✔ | ✔ | X | ✔ | ✔ | ✔ |
| PDX | X | X | X | ✔ | X | X | X | ✔ | ✔ | X | ✔ | ✔ | ✔ |
| GOS | X | X | X | ✔ | X | X | X | ✔ | ✔ | X | ✔ | ✔ | ✔ |
| DHA | X | X | X | X | ✔ | ✔ | X | ✔ | ✔ | X | ✔ | ✔ | ✔ |
| ARA | X | X | X | X | X | ✔ | X | ✔ | ✔ | X | ✔ | ✔ | ✔ |
| HMO | X | X | X | X | X | X | ✔ | X | ✔ | X | X | ✔ | ✔ |

Key: ✔ = present; X = not present

1. A method for the treatment and/or prevention of 2019 novel coronavirus (COVID-19) infection in a subject, wherein the method comprises the step of administering lactoferrin to the subject.

2. The method of claim 1, wherein the daily dosage of lactoferrin is in the range of about 1 mg/day to about 50 g/day.

3. The method of claim 1 , wherein the lactoferrin is in the form of a reconstituted solution, preferably wherein the reconstituted solution comprises lactoferrin in the range of about 0.1 mg/mL to about 0.25 g/mL.

4. The method of claim 1, wherein the lactoferrin is a COVID-19 immunologic adjuvant.

5. (canceled)

6. The method of claim 4 , wherein the lactoferrin is provided to the subject before, during, and/or after COVID-19 vaccine administration.

7-9. (canceled)

10. A method for the treatment and/or prevention of 2019 novel coronavirus (COVID-19) infection in a subject, wherein the method comprises the step of administering a composition to the subject, and wherein the composition comprises lactoferrin.

11. The method of claim 10, wherein the composition comprises lactoferrin in the range of about 15 mg/100 kcal to about 25 g/100 kcal.

12. The method of claim 10 , wherein the composition is a nutritional composition.

13. The method of claim 10, wherein the composition is intended for a paediatric subject.

14. The method of claim 13, wherein the lactoferrin is present in the range of about 15 mg/100 kcal to about 300 mg/100 kcal.

15. The method of claim 10, wherein the composition is intended for an adult.

16. The method of claim 10, wherein the composition is a synthetic composition.

17. A method for the treatment and/or prevention of 2019 novel coronavirus (COVID-19) infection in a subject, wherein the method comprises the step of administering a pharmaceutical composition to the subject, and wherein the pharmaceutical composition comprises lactoferrin.

18. The method of claim 17, wherein the daily dosage of lactoferrin is in the range of about 1 mg/day to about 50 g/day.

19. The method of claim 17 , wherein the pharmaceutical composition comprises at least one pharmaceutically acceptable excipient, preferably the at least one pharmaceutically acceptable excipient comprises a binder, a disintegrant, a diluent, a glidant, a solubilizer, a lubricant, a flavouring agent, any other pharmaceutically acceptable excipient, or a combination thereof.

20. The method of claim 17, wherein the pharmaceutical composition is a COVID-19 immunologic adjuvant.

21. (canceled)

22. The method of claim 20 , wherein the pharmaceutical composition is provided to the subject before, during, and/or after COVID-19 vaccine administration.

23. The method of claim 22, wherein the pharmaceutical composition is provided to the subject immediately prior to the COVID-19 vaccine administration, provided on the day of the COVID-19 vaccine administration but prior to COVID-19 vaccine administration, provided for at least one day prior to the COVID-19 vaccine administration, provided for at least two to four days prior to the COVID-19 vaccine administration, provided for at least five to seven days prior to the COVID-19 vaccine administration, provided for at least eight to fourteen days prior to the COVID-19 vaccine administration, provided for at least fifteen to twenty-nine days prior to the COVID-19 vaccine administration, provided for at least thirty to fifty-nine days prior to the COVID-19 vaccine administration, provided for at least sixty to eighty-nine days prior to the COVID-19 vaccine administration, or provided for at least ninety or more days prior to the COVID-19 vaccine administration.

24. The method of claim 22, wherein the pharmaceutical composition is provided to the subject immediately after the COVID-19 vaccine administration, provided on the day of the COVID-19 vaccine administration but after COVID-19 vaccine administration, provided for at least one day after the COVID-19 vaccine administration, provided for at least two to four days after the COVID-19 vaccine administration, provided for at least five to seven days after the COVID-19 vaccine administration, provided for at least eight to fourteen days after the COVID-19 vaccine administration, provided for at least fifteen to twenty-nine days after the COVID-19 vaccine administration, provided for at least thirty to fifty-nine days after the COVID-19 vaccine administration, provided for at least sixty to eighty-nine days after the COVID-19 vaccine administration, or provided for at least ninety or more days after the COVID-19 vaccine administration.

25-31. (canceled)
